(19) United States
(12) Patent Application Publication
(10) Pub. No.: US 2008/0146870 A1
(43) Pub. Date: Jun. 19, 2008

(54) SYSTEM AND METHOD FOR CORRECTING ERECTILE DYSFUNCTION

(76) Inventor: John L. Marchello, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/610,827

(22) Filed: Dec. 14, 2006

(51) Int. Cl. A61F 5/41

(52) U.S. Cl. 600/38

(57) ABSTRACT

A therapeutic device used to treat erectile dysfunction helps to maintain tissue flexibility and encourage blood flow into the penis, thereby aiding in the healing process. The article is a unitary, molded product using a flexible, resilient material such as transparent or translucent medical-grade vinyl. The article is a generally bullet-shaped bulb having a proximal opening that transitions into a penile expansion chamber through a necked-down neck/seal section. The penile expansion chamber is a dual-purpose section activated by a user's hands to create a soft vacuum which draws the penis into the chamber and retains it. A flange area surrounding the proximal opening provides a comfortable surface for support of the article against the abdomen of the user, and does not become part of the air/vacuum seal. The neck/seal area forms an airtight seal as the penis expands.

### FIELD OF THE INVENTION

This invention relates to the treatment of erectile dysfunction and, in particular, to a flexible, resilient bullet-shaped bulb that helps to maintain tissue flexibility and encourage blood flow into the penis.

### BACKGROUND OF THE INVENTION

Erectile dysfunction (ED) is the inability of a man to achieve or maintain an erection. Most men experience this at some point in their lives, usually by age 40, and are not psychologically affected by it. Some men, however, experience chronic, complete erectile dysfunction (impotence), and others, partial or brief erections. Frequent erectile dysfunction can cause emotional and relationship problems, and often leads to diminished self-esteem. Erectile dysfunction has many causes, most of which are treatable, and is not an inevitable consequence of aging.

### SUMMARY OF THE INVENTION

This invention resides in a therapeutic device used to treat erectile dysfunction. The device helps to maintain tissue flexibility and encourage blood flow into the penis, thereby aiding in the healing process.

In the preferred embodiment, the article is a unitary, molded product using a flexible, resilient material such as transparent or translucent medical-grade vinyl. The article is a generally bullet-shaped bulb having a proximal opening that transitions into a penile expansion chamber through a necked-down neck/seal section. The penile expansion chamber is a dual-purpose section activated by a user's hands to create a soft vacuum which draws the penis into the chamber and retains it. A flange area surrounding the proximal opening provides a comfortable surface for support of the article against the abdomen of the user, and does not become part of the air/vacuum seal. The neck/seal area forms an airtight seal as the penis expands.

The article may be used in conjunction with other treatments for erectile dysfunction, including 1) oral medications, such as Viagra, Levitra, etc.; 2) other vacuum therapy devices; 3) penile self-injection; and 3) transurethral insertion therapy. Use of the article prior to any of the above treatments will generally act to enhance the treatment's results due to increased blood flow and tissue elasticity.

### BRIEF DESCRIPTION OF THE DRAWINGS

FIG. 1 is an oblique, perspective view of the preferred embodiment of the invention;

FIG. 2A is side view of the preferred embodiment

FIG. 2B is a front view;

FIG. 2C is a rear view; and

FIG. 3 is a side view cross section with dimensions.

### DETAILED DESCRIPTION OF THE PREFERRED EMBODIMENTS

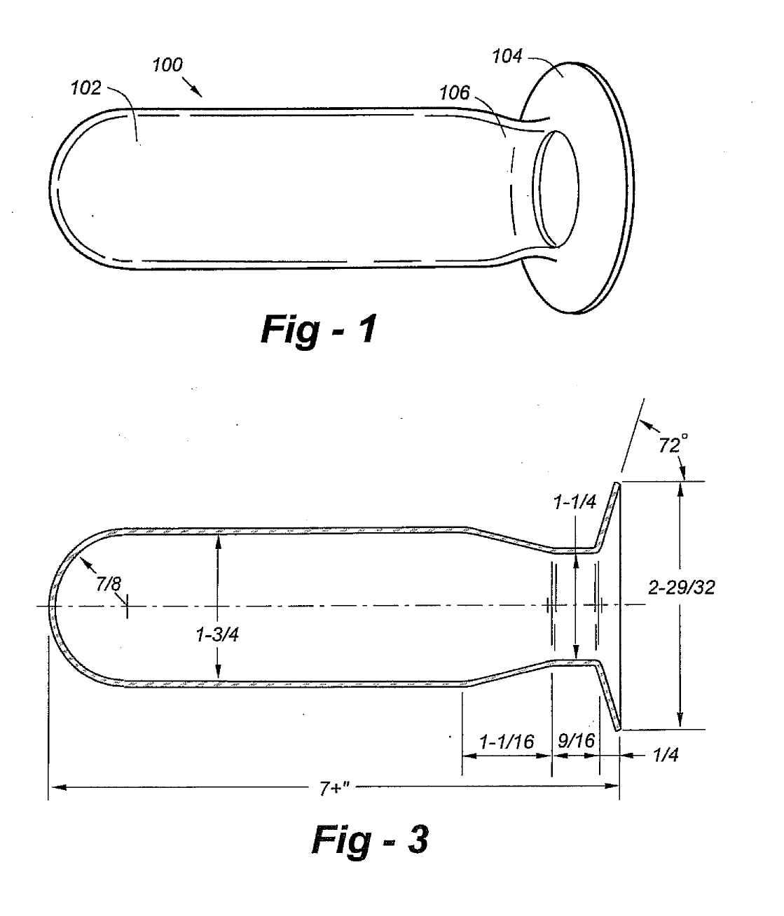

FIG. 1 is an oblique, perspective view of the preferred embodiment of the invention depicted generally at 100. The penile expansion chamber 102 is a dual-purpose section activated by a user's hands to create a soft vacuum which draws the penis into the chamber and retains it. The flange area 104 provides a comfortable surface for support of the article against the abdomen of the user, and does not become part of the air/vacuum seal. The neck/seal 106 area forms an airtight seal as the penis expands. This maintains a vacuum within the chamber allowing blood to flow easily through the penile arteries and into the corpora cavemosa, thus creating the tumescent or swollen penis.

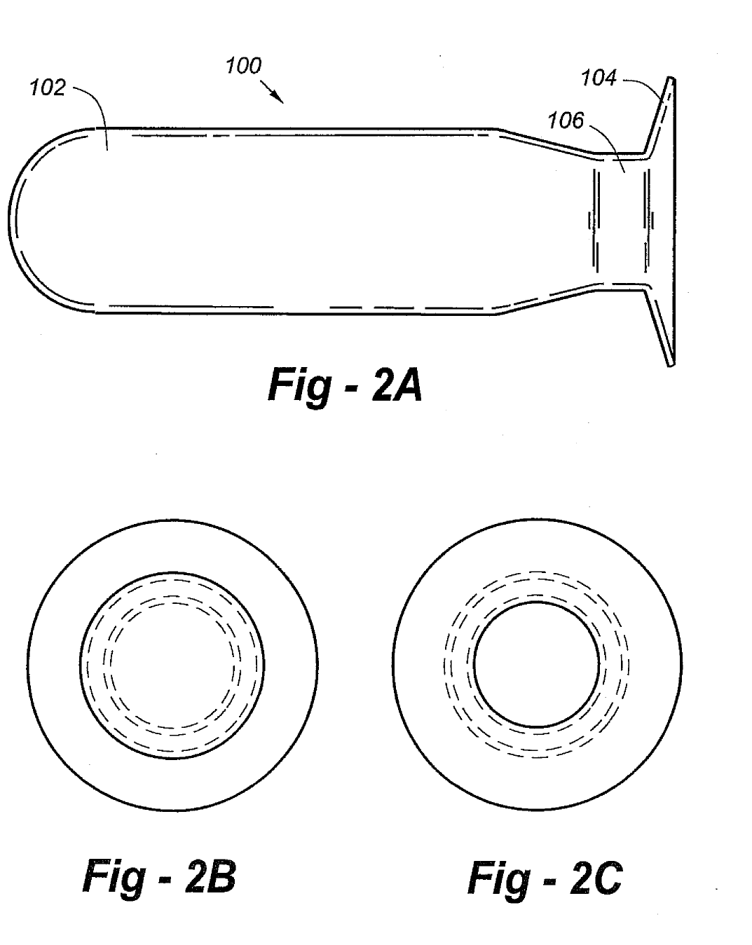

The article is preferably constructed as a unitary molded product using a flexible, resilient material such as medical grade vinyl having a wall thickness on the order of 0.100 inches and a weight in the range of 90-100 grams. Clear or translucent material is preferred. FIG. 2A is side view of the preferred embodiment, FIG. 2B is a front view, and FIG. 2C is a rear view.

Figure is a side-view cross section with dimensions. The overall length is 5 inches or greater, preferably 7 inches or more. The neck/seal 106 area features a proximal opening with an inner diameter in the range of 1 to 1.5 inches, preferably 1.25 inches. The proximal opening transitions into the penile expansion chamber 102, which has a diameter in excess of 1.5 inches, preferably 1.75 inches.

In terms of use, lubrication should be spread around the proximal opening of the center of the flange area and into the interior of the penile chamber as far as the forefinger can reach. Lubricating will prevent irritation to the glans (head) of the penis and also act as a seal between the base of the penis and the neck/seal area. When using the article in the tub or shower, petroleum jelly can be used, as it does not wash off. Oil-based lubricants will not damage the article. Lubrication should also be spread on the glans (head) of the penis.

The user should wipe any excess lubricant off their fingers to insure a good grip and, using both hands, compress the article as completely as possible. The opening of the penile chamber is positioned snugly against the glans (head) of the penis as the article is drawn towards the abdomen. The hands are then opened, allowing for expansion of the chamber. This action should draw the penis into the chamber and increase blood flow into the penis. This step may have to be repeated a few times so that enough blood flows into the penis to expands its circumference, so that a seal occurs between the base of the penis and the neck/seal area.

When a seal is achieved around the base of the penis, a soft vacuum should remain inside the chamber. This vacuum will slowly continue to draw blood into the penis, expanding its circumference. The user should allow approximately 60 seconds for this blood flow to occur. To use the article for penile therapy, it can be activated in one of two ways or by using a combination of both actions. The user may gently push/pull the article backward and forward over the shaft of the penis with approximately one inch of movement. This stroking will stretch the penis to increase tissue elasticity forcing blood through the corpora cavernosa. The user may intermittently pause from stroking and compress the penile expansion chamber against the penis firmly, so that the penis flattens. If held and released several times, this action will force blood out of the corpora cavernosa, returning the blood to the penile veins and back to the body. Combinations of stroking and compression can be used depending on the user's hand strength and comfort level. No pain should be experienced during these therapy sessions,

I claim:

1. An article for treating erectile dysfunction, consisting of:
a hollow bulb composed of a flexible, resilient material having a length of 5 inches or greater,
the bulb including a proximal opening with an inner diameter in the range of 1 to 1.5 inches; and
wherein the proximal opening transitions into an interior volume having a diameter of over 1.5 inches.

2. The article of claim 1, wherein the material is medical-grade vinyl.

3. The article of claim 1, wherein the material is semi-transparent or transparent.

4. The article of claim 1, further including a peripheral flange surrounding the proximal opening

5. An article for treating erectile dysfunction, consisting of:
a hollow bulb composed of a flexible, resilient, gas-impermeable material having a length of 5 inches or greater
the bulb including a proximal opening with an inner diameter in the range of 1 to 1.5 inches;
a peripheral flange surrounding the proximal opening; and
wherein the proximal opening transitions into an interior volume having a diameter of over 1.5 inches.

6. The article of claim 5, wherein the material is medical-grade vinyl.

7. The article of claim 5, wherein the material is semi-transparent or transparent.

8. An article for treating erectile dysfunction, comprising:
a hollow bulb composed of a flexible, resilient, gas-impermeable material having a length of 5 inches or greater,
the bulb including a proximal opening with an inner diameter in the range of 1 to 1.5 inches;
a peripheral flange surrounding the proximal opening; and
wherein the proximal opening transitions into an interior volume having a diameter of over 1.5 inches.

9. The article of claim 8, wherein the material is medical-grade vinyl.

10. The article of claim 8, wherein the material is semi-transparent or transparent.

11. A method of treating erectile dysfunction, comprising the steps of:
providing the article of claim 1;
inserting a penis through the proximal opening; and
manually manipulating the bulb to establish a vacuum in the interior volume.

12. The method of claim 10, further including the step of providing a lubricant around the proximal opening prior to insertion of the penis.

13. A method of treating erectile dysfunction, comprising the steps of:
providing the article of claim 8;
inserting a penis through the proximal opening until the flange contacts the user, thereby creating a seal around the proximal opening; and
manually manipulating the bulb to establish a vacuum in the interior volume.

14. The method of claim 123, further including the step of providing a lubricant around the proximal opening prior to insertion of the penis.
